(19) United States
(12) Patent Application Publication
(10) Pub. No.: US 2012/0238972 A1
(43) Pub. Date: Sep. 20, 2012

(54) MEDICAL SUCTION DEVICES AND METHODS OF USE

(75) Inventors: John Karpowicz, Chester Springs, PA (US); Kevin Klocek, Wynnewood, PA (US); Thomas Ward, Coatesville, PA (US)

(73) Assignee: Boehringer Laboratories LLC, Phoenixville, PA (US)

(21) Appl. No.: 13/416,261

(22) Filed: Mar. 9, 2012

Related U.S. Application Data
(60) Provisional application No. 61/453,792, filed on Mar. 17, 2011.

(51) Int. Cl. A61M 1/00

(52) U.S. Cl. 604/319

(57) ABSTRACT

Devices for controlling the level of suction to a patient in a hospital suction collection system are disclosed. The hospital collection system includes a conduit arranged to be coupled to the patient and a wall plate coupled to a source of suction and to which the device will be connected. The device includes a plastic body, an inlet portion, an outlet portion and a regulator for sensing and controlling the level of suction applied to the inlet portion. Means may be provided for ensuring that when the device is subjected to excessive mechanical force it operates in a predetermined manner, e.g., fails or pulls out of the wall plate. The device may includes means to ensuring that after it is disconnected from the wall plate it cannot be reconnected to it and a fluidic dampening system to dampen oscillatory vibrations.

### CROSS-REFERENCE TO RELATED APPLICATIONS

This application claims priority from Provisional Application Ser. No. 61/453,792, filed on Mar. 17, 2011, entitled Medical Suction Devices and Methods of Use, which application is assigned to the same assignee as this application and whose disclosure is incorporated by reference herein.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### FIELD OF THE INVENTION

The disclosed invention relates to devices for a medical fluid aspiration apparatus that includes suction control mechanisms and clinical safety mechanisms for medical purposes and methods of use.

### BACKGROUND OF THE INVENTION

Suction controls are routinely used in hospitals to regulate the level of suction delivered from a central supply system to the patient. Patient safety requires the high vacuum from the central supply be regulated to a lower safe level and adjustable for any number of clinical needs. On its most basic level, suction is used to remove fluids and debris from body cavities and is employed in virtually any location where a patient is present. Modem hospitals employ central suction systems with distributed supply at the patient bed. Mechanical controls reduce the high vacuum levels present in the central suction system to lower levels safe for patients and suitable for the patient collection devices. Collection devices are typically plastic, disposable devices that are used to accumulate fluids and debris. The collection devices are located between the point of collection and the control device and serve to protect the piping system and manual controls from contamination by bodily substances.

The attachment to hospital wall suction outlets is accomplished via a variety of quick-connect fittings. These quick connects vary in design and effectiveness and are typically metallic in their construction.

The manual controls that interface high vacuum in the central suction system with the patient contact collection systems are typically reusable, mechanical devices. The construction of these devices makes them prone to retention of fluids that may harbor infectious agents. Unlike other fluidic systems in the hospital, such as oxygen, nitrogen, or medical air, the inlet to the suction system is proximal to the patient and may not be filtered. In addition to room air, caregivers unintentionally introduce foreign material into the flow paths of these devices. The devices also contribute to contact vectors for contamination.

A complete fluid aspiration device consists of a control or regulator that attaches to the hospital central suction system for applying safe levels of suction pressure, collection devices for the accumulation of fluids and air separation, tubing and patient contact items, such as a catheter or a collection wand. These elements do not exist as a single item and need to be assembled at the point of care. In an emergency, this can be time critical since there may be an acute need to remove fluids from a patient's breathing passages. Additionally, the user needs to make adjustments and settings that also take time away from more critical activities.

One primary clinical use for emergency suction is to clear the airway of a patient that has aspirated fluids and materials. The airway must be rapidly cleared prior to initiating any ventilation of the patient to avoid driving fluid and materials into the lungs. This clearing process must also employ suction pressures and techniques that avoid damaging the sensitive mucosal tissues in a patient's airway. Often, the act of having to reach back to the control to make adjustments is simply not practical in an emergency.

Other uses for suction occur in a surgical environment and the need is to maintain a clear field to visualize the operative site. High levels of suction are routinely employed in these cases because there is the ability for a medical professional to intervene and remove the suction apparatus should it come into contact with sensitive tissue.

Emergency suction is also employed on individuals that spontaneously arrive at the hospital for the reasons stated above. There is little time to assess whether these patients have a communicable disease. Suction equipment is particularly affected by such patients due to the nature of contact with compromised tissue. There is an accepted standard called the Spaulding classification that identifies suction controls as semi-critical in some applications and therefore require some form of sterile processing of this equipment following use. Primary collection vessels and tubing are routinely discarded following patient use, but the control devices are not. In a busy emergency room, a suction control could be used many times before it was determined that a prior patient was infected with a communicable disease. It would be preferable to be able to dispose of these control devices and ensure that they are not inadvertently used in other applications.

Conventional prior art suction regulators are typically constructed to provide adjustable suction levels based on individual clinical requirements. Essential features for a suction regulator include an inlet and outlet for attachment to the patient and a source of suction; a control mechanism for sensing changes in flow demand conditions and controlling the suction pressure output; a gauge for indicating the controlled output and specific and clinical safety measures that are specific to the type of suctioning to be performed.

Suction control mechanisms must provide a means of “sensing” the delivered suction pressure and making adjustments to the sealing mechanism as changes to conditions in the collection circuit occur. The ‘sensing’ function is accomplished by allowing a feedback signal of air or fluid to communicate with the sensing side of the diaphragm. This feedback traditionally exposes the control mechanism to foreign materials and fluids that are introduced into the circuit. The control mechanism traditionally consists of a diaphragm opposed by a spring. The sensing side of the diaphragm isolates the control surface from an atmospheric reference. An increase in suction pressure on the sensing or working side of the diaphragm results in a change in relative position of the diaphragm such that a sealing surface can engage and shut off the source of suction. One such regulating mechanism is described in U.S. Pat. No. 7,686,785 whose disclosure is incorporated by reference herein.

As will be appreciated by those skilled in the art suction devices should provide regulated suction in a specified range through the full range of clinically necessary flows. The control must not stick especially after periods of storage and there should be no vibration or buzzing across the full range of flows that are encountered clinically.

Suction controls typically incorporate an indicating mechanism that clearly indicates the level of applied suction. One such gauge or indicating mechanism is described in U.S. Pat. No. 5,992,239 and is herein incorporated by reference. The indicating mechanism or gauge must have suitable accuracy for the clinical application and be easy to interpret by clinical staff. The gauge also must be protected from intrusion by foreign materials that could affect its function and accuracy as well as being an area that harbors infectious materials. A disposable device need not incorporate a gauge mechanism when used for a specific clinical application wherein adjustment and verification are unnecessary. The control device must be reliable in its delivery of said suction to allow elimination of this feature.

Suction controls may require a safety mechanism that is unique to the clinical application. Gastric drainage for example employs an intermitting cycling of the suction pressure to guard against continual application of suction to the interior of the gastric space which could result in traumatic lesions and possibly bleeding. Emergency suction requires that the clinical intervention be accomplished as quickly as possible and permit suctioning maneuvers without requiring the clinician to make adjustments at the wall but will permit point of use emergency application in cases of extreme need.

As will be described below the devices of this invention address many problems and needs of the prior art. Each device of this invention is arranged to deploy effortlessly into the hospital quick connection system. Due to the possibility that a device will become contaminated with bacteria, the device has mechanisms to prevent reuse. Moreover, the device incorporates a lockout mechanism, such that once the device is placed into the wall receptacle it can never be placed in another wall receptacle for use on another patient, thus protecting subsequent patients and staff from exposure to potentially infectious materials.

### SUMMARY OF THE INVENTION

A device for controlling the level of suction to a patient in a hospital collection system having receptacle member, e.g., a wall plate, coupled to a source of suction in the hospital collection system. The hospital collection system includes a conduit arranged to be coupled to the patient to apply suction to the patient. The device basically comprises a body, an inlet portion and an outlet portion. The inlet portion is arranged for connection to the conduit to the patient. The outlet portion is arranged for releasable connection to the receptacle member to couple the device to the source of suction of the hospital collection system to the patient. The body of the device comprises a regulator for sensing and controlling the level of suction applied to said inlet portion.

In accordance with one aspect of this invention the device includes means for ensuring that when the device is subjected to excessive mechanical force it operates in a predetermined manner. For example, one aspect of the predetermined manner comprises the outlet portion failing. Another aspect of the predetermined manner comprises the device pulling out of the receptacle member.

In accordance with another aspect of this invention the device includes a fluidic dampening system to dampen oscillatory vibrations.

In accordance with another aspect of this invention the device includes means for ensuring that when disconnected from said receptacle member it cannot be reconnected thereto, e.g., the device is arranged for single use.

### DESCRIPTION OF THE DRAWING

FIG. 1 is a side view of one exemplary embodiment of a device constructed in accordance with this invention for releaseable securement to a wall plate of a hospital suction collection system, with the left hand portion of the figure representing the device, the right hand portion of the figure representing the wall plate and associate collection system, and with the portion of figure at its bottom representing an enlarged view of a portion of the device shown in the left hand portion of the figure;

FIG. 2 is a side view of a latch design quick connect feature of the device shown in FIG. 1, with the left hand portion of FIG. 2 representing a prior art metal quick connector, and with the right hand portion representing one exemplary embodiment of a plastic quick connector constructed in accordance with this invention;

FIG. 3 is an enlarged side view of an exemplary embodiment of a device constructed in accordance with this invention showing its break away feature;

FIG. 4 is a side view of another exemplary embodiment of a device constructed in accordance with this invention and making use of a modular quick connect system, with the lower portion of the figure being in cross section to show the corresponding portion of the device shown in the upper portion of the figure;

FIG. 5 is a cross sectional view of one exemplary embodiment of a device constructed in accordance with this invention;

FIG. 6 is an enlarged cross section of an internal portion of the device shown in FIG. 5; and

FIG. 7 is an enlarged cross section of an internal portion of an alternative device constructed in accordance with this invention.

### DETAILED DESCRIPTION OF THE PREFERRED EMBODIMENT

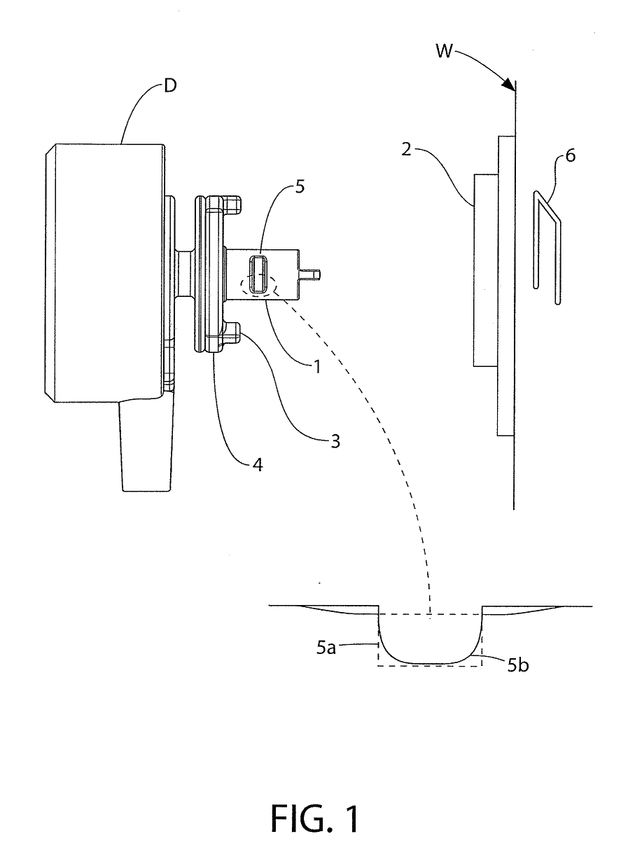

Referring to FIG. 1, port 1 of device D inserts into wall W via a receptacle member or wall plate 2. The wall plate forms a portion of the patient collection circuit of the hospital's suction-based collection system. There are a number of different wall plate designs for medical gases such as the DISS (Diameter Index Safety System). The DISS system is an internationally recognized system that employs unique diameters and threads for distinguishing different types of gases. There are also proprietary systems produced by independent organizations, such as the Diamond quick-connect system as produced by Ohio Medical Products. Allied Healthcare produces the Chemetron quick connect system and there are others produced by Puritan-Bennet, Schrader and others. DISS, Diamond and Chemetron are the most popular and would encompass a majority of uses.

The quick connect illustrated in FIG. 1 is of the Diamond variety and plugs into wall receptacle 2. Tabs 3 on ring 4 are positioned for a specific medical gas and provide the assurance that this device is only applied to a suction inlet. The tabs are oriented to the retention groove 5. The conventional Diamond quick connect incorporates a spring return mechanism for release. The suction device D of this invention eliminates this spring return feature. To release the device D, the entire device is rotated, in so doing ring 3 is displaced from its starting position. Once displaced, the unit can not be reinserted into the wall plate 2 because the locating tabs 3 no longer line up with the retention groove 5 thus providing a one time insertion removal cycle. The one time feature can be overridden with a tool or special procedure if needed. Similar one time mechanisms are anticipated for the DISS and Chemetron fittings.

The Diamond style quick connect is retained in the wall fitting through the use of a wire retention 6 that snaps into a groove 5. The groove in the conventional mechanism is a square groove as shown by dotted line 5a. In contradistinction, the groove 5b of this invention is radiused to provide the maximum available strength, since the device of this invention is preferably formed of a plastic material. This radius 5b minimizes stress and additionally is positioned such that the wire retention 6 will penetrate the groove by more than one half of the wire diameter, thus ensuring that the device will not cam out unnecessarily. Similar stress relieving designs are anticipated for the Chemetron and DISS style of quick connections.

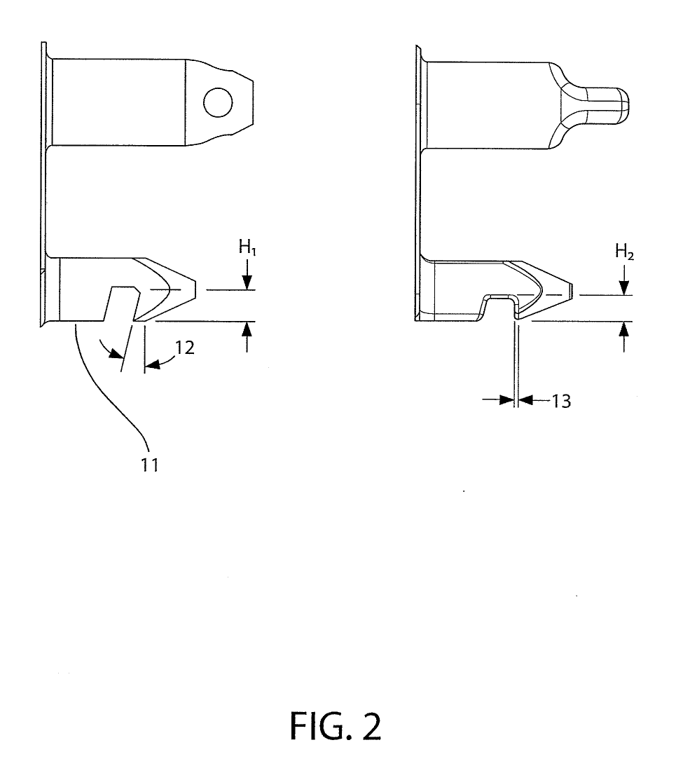

The Chemetron style of quick connect is shown in the left hand portion of FIG. 2 and has a port that inserts into the wall receptacle and is retained by a latch component 11. The latch is a machined groove that has a back relief angle 12 cut into it. This relief angle is problematic for a plastic or molded element in that it reduces the strength and upon breakage would result in leaving a small piece of plastic inside of the wall connector. Thus, the quick connect of the subject invention, which is shown in the right hand portion of FIG. 2, is modified, so that the strength of the latch is greatly improved by reducing the relief angle 13 to near zero and shortening the depth H2 to a value less than H1 on the metal version. This change improves the strength of the latch to the point where the device will pull out of the wall outlet prior to breakage.

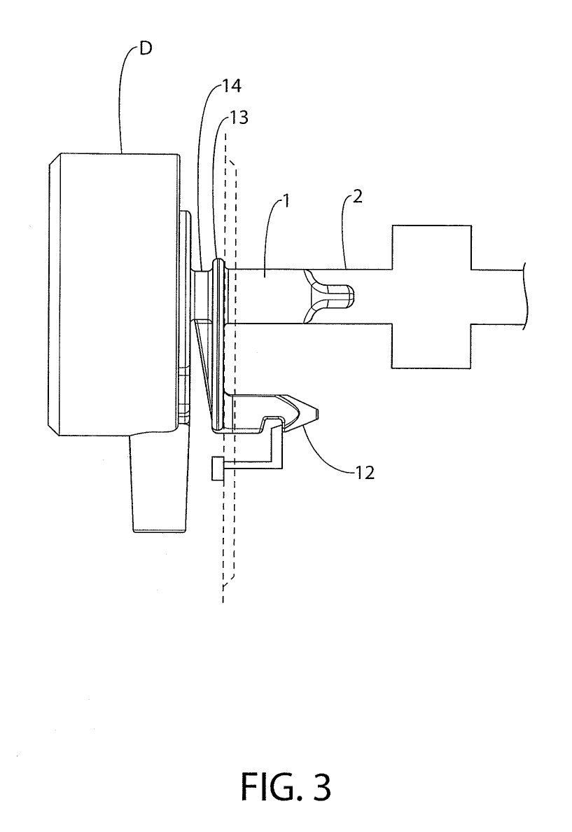

Referring now to FIG. 3, a common condition associated with all devices that are inserted to hospital wall fittings is abuse and breakage due to a variety of conditions, such as being hit with a bed or other piece of equipment. Other equipment can fall against the device. Staff can pull on tubing causing breakage as well. The metal version of the wall quick connects experience damage, so it is a concern that plastic versions would be more susceptible to breakage. While it is not practical to eliminate breakage due to random accidental events, it is possible to ensure that if breakage occurs, then the remnant components are readily removed from the wall allowing efficient replacement with a new unit. The device illustrated in FIG. 3 is a device with the Chemetron quick-connect system. Other quick-connect systems would lend themselves to the following approach. Device D is placed into wall connection 2. Fitting 1 provides the pathway for fluid flow and the latch mechanism 12 retains the device into the wall fitting. The area behind the flange 13 is a controlled diameter or necked down area 14, such that should there be a shearing, twisting, torque force that could cause breakage, then the failure occurs at the necked down area of the device. The end result of this breakage leaves a remnant component that can be readily removed from the wall fitting 1. The necked down area functions as a fuse of sorts permitting quick removal of the remnant and return of service via replacement of the entire device.

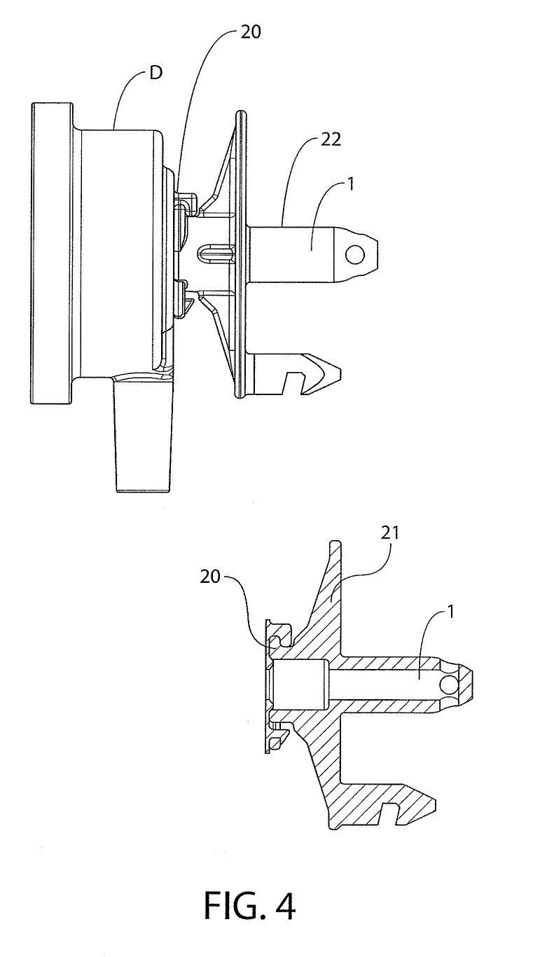

Referring now to FIG. 4, a further embodiment of a device D of this invention is shown. That device incorporates a modular connection where the various quick connect designs can be modularly attached to the base device. Shown is device D with the Diamond version 22 attached at point 20. Also shown is a Chemetron version 21 that can readily replace the Diamond version by twisting the Diamond version off and applying the Chemetron version. Other versions of this interconnect can be readily imagined using a slide mechanism etc. This feature provides a hospital the flexibility to adapt to any type of wall fitting should there be different types available in the facility. Note that the fuse mechanism previously described is contained in this embodiment as well.

It is also important to maintain as large a flow path as possible through port 1 in all quick connect configurations. Minimizing stress risers through the use of radius grooves ensures that the maximum internal dimensions are maintained. The devices described herein maintain an internal diameter in the flow passage greater than 0.125″ and preferably greater that 0.2″ to provide superior flow while still maintaining suitable strength of the components in actual use.

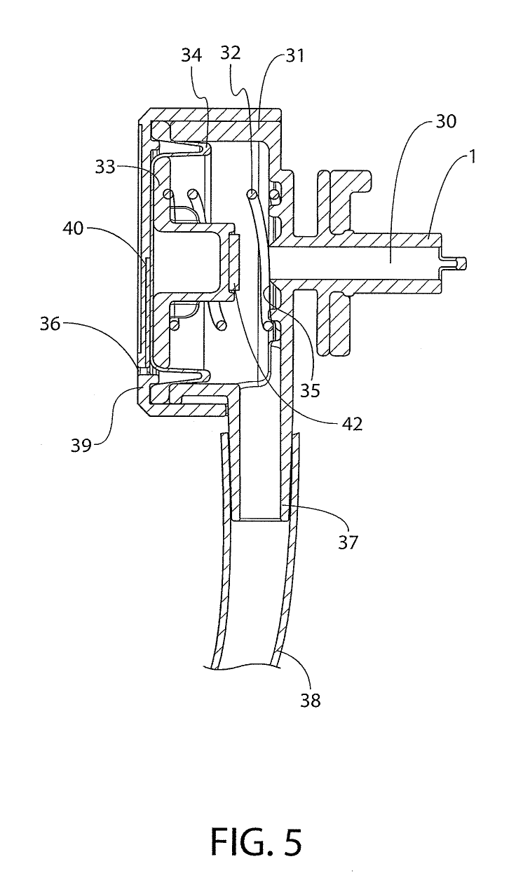

Referring now to FIG. 5, upon insertion into wall fitting 2, suction is applied to the device D via the internal conduit 30 of port 1 to the interior of a regulating mechanism housing 31. In a preferred embodiment of this invention, it is desired to provide a safe controlled level of suction for clinical applications where the requirements on the clinician are minimized to the essential minimum tasks. The regulating mechanism as depicted in the cross section of FIG. 5 provides a fixed suction output via a spring 32 opposing a piston 33 that is sealed by a rolling style diaphragm 34. Rolling diaphragms are available from Bellofram Inc. The rolling diaphragm configuration provides a low friction control feature which allows the seal 42 on the end of the piston 33 to approach a sealing surface 35 in the body portion 31 of the regulator under the influence of suction pressure. The output suction is determined by the effective rate of the spring 32 and the effective area of the diaphragm 34 as well as a reference to atmospheric pressure that is provided by vent 36. This mechanism causes a sealing engagement of the end of the piston 33 with the sealing surface 35 at the desired suction pressure output. Any reduction in the suction pressure at the inlet condition causes the spring 32 to move the piston 33 away from the sealing surface 35 thus increasing the suction flow and pressure until the set-point is restored.

Output suction is delivered via port 37 to tubing 38 that is subsequently attached to collection devices such as canisters, collection wands etc. (not shown).

The nature of a disposable device is such that it needs to provide consistent suction upon use even after extended periods of storage. To that effect, a potential problem would exist should there be any sticking of diaphragm 34 to the inside surface of cover 39. Special treatments are employed such as non-stick coatings (i.e., Teflon, talc, silicone release agents) on the surfaces that are maintained in intimate contact. Additionally, grooves or slots 40 are employed in cover 39 to allow communication of the atmospheric reference air to enter the space between the diaphragm 34 and cap 39.

High flow through the mechanism is maintained not only through the use of large bore passages as previously described for the quick connect portion of the device, but through the careful configuration of the spring 32 diaphragm 34 combination and their relationship to the sealing area 35. The change in the flow passage as flow transitions from the internal conduit 30 of the ports to the internal chamber 41 of the mechanism housing 31 is important in the design of a regulator.

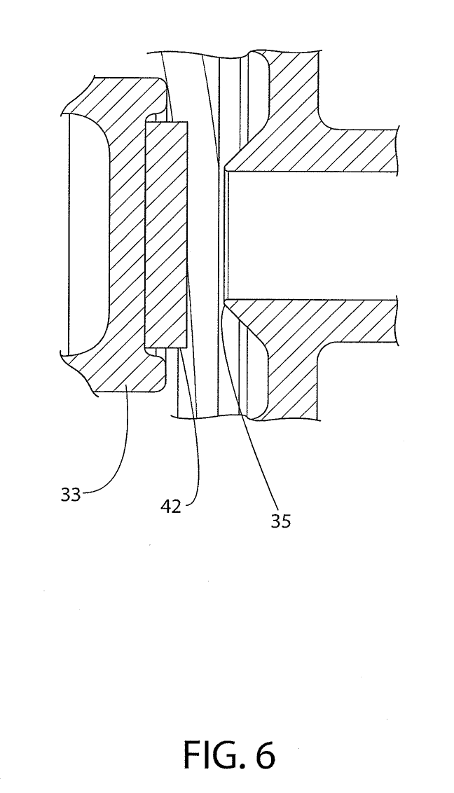

Referring to FIG. 6, the algebraic expression that illustrates the functional aspect of flow is obtained by equating the cross sectional area D of the conduit 30 to the area of cylinder defined by a fully opened passage as follows:

Π*²/4=Π*D*h

Reducing this expression yields

H=D/4

Consequently for a 0.2″ conduit, providing an opening of 0.2″/4 or 0.05″ will yield a control mechanism with minimal pressure drop while maintaining superior flow. An optimal control will provide a diaphragm/spring combination that will provide at least D/4 movement with as low a change in pressure as possible, typically 10% of set-point or better. The spring diaphragm combination in use in this device will provide D/4 movement for a change in circuit pressure of 10% of setting. This characteristic ensures that the device regulates effectively across the full range of flows experienced from zero flow in a dead ended or occluded condition to full flow for emergency aspiration in the range of 100 liters per minute. It is acceptable to have a regulator that will control output pressure to within 20% of the set-point. It is preferred to maintain better than 10%.

Output suction pressures for this device are changed by changing the rate of spring 32 or by changing the relative position of sealing surface 35 with the seal 42 at the end of the piston 33 to provide sealing engagement at a greater or lesser spring force.

### Buzzing

A common problem with pneumatic controls results in fluidic vibration such that at certain flows or pressures, there is a vibration induced by flow across the sealing surface that results in audible vibration or buzzing. This problem is significant. It is often dealt with by incorporating mechanisms that induce a low level of friction that tends to cancel the noise. The subject invention has eliminated the complex mechanisms employed for this and other purposes and as such, there is need to incorporate other noise canceling methods. The subject invention addresses noise by incorporating a fluidic dampening system on the atmospheric side of diaphragm 34. A controlled vent 36 restricts the flow of air into the reference chamber and effectively eliminates vibration. The size of the vent is important and it has been found that for the current size of the device, a vent in the range of 0.045″ to 0.07″ is optimal and 0.03″ to 0.12″ will function. Less than 0.03″ results in an over damped control where response is sluggish. Greater than 0.07″ results in under dampening and excessive overshoot of setting. Greater than 0.12″ results in the onset of vibration.

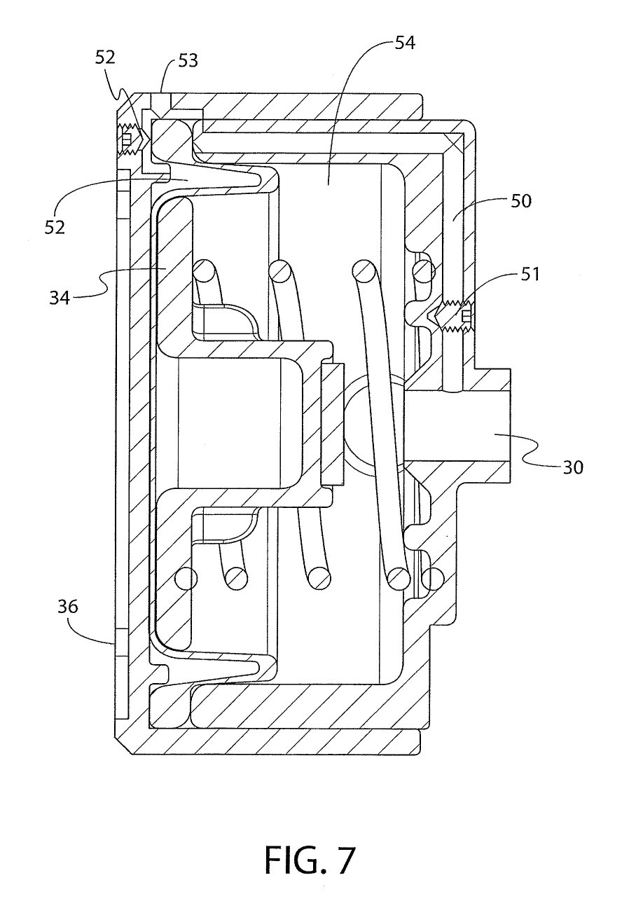

Attention is now directed to FIG. 7 where a further embodiment and refinement are described. In addition to a fixed output device that is the subject of this invention, a variable output device can be configured via the manipulation of the atmospheric vent in conjunction with a pilot signal. A high pressure pilot signal is directed from internal conduit 30 via pilot passage 50 to the atmospheric side of diaphragm 34 referred to as a control chamber. Relative pressures to the control chamber can be varied/adjusted via fixed restrictors 51 and 52. Pilot passage 50 is extended out of the device via tube 53. Complete or partial occlusion of tube 53 results in a change in the pressure in the control chamber 52. As the suction pressure in the control chamber 52 exceeds the suction pressure in the regulated chamber 54, the diaphragm mechanism is urged to a more open position resulting in an increase in pressure. Thus this embodiment enables rapid increase in output suction for any application requiring same by simply occluding a tube or port. The port can be positioned right on the device or can be remotely located on a suction instrument right in the hand of the clinician.

Removing the occlusion from the port returns the device to the preset safe level. Partially occluding the pilot port will result in elevated, but not necessarily full suction power and conversely applying a positive pressure will turn off suction.

The pilot flow and spring arrangement can be configured to provide a preset condition from no suction to any predetermined level and occlusion of the pilot can give complete control of applied suction from zero to full suction. These capabilities put the control right in the hands of the clinician performing the maneuver eliminating the time consuming and potentially life threatening steps required with conventional devices.

It is also possible through the use of controlled pilots to eliminate the spring entirely and have controlled pilots deliver suction pressures from zero or off to a controlled level to the maximum available at the wall outlet.

As should be appreciated by those skilled in the art from the foregoing, the subject invention enables the rapid deployment of suction in a clinical environment that provides a disposable suction regulator incorporating a quick connection for attachment to hospital wall suction inlets, wherein the quick connection incorporates a single use feature. The devices make use of quick connection features which are arranged for attachment to hospital wall suction inlets and that replace conventional metal components with plastic. Moreover, they are configured such that upon abuse, the plastic elements fail in a desired manner as to not obstruct the wall fitting. Moreover, the devices of this invention employ quick connections which modularly convert to differing styles. Further still, the quick connections are arranged for attachment to hospital wall suction inlets wherein they are effectively retained by the wall system, but release in a predefined manner when subjected to abuse.

Devices in accordance with this invention may constitute fixed output regulating mechanisms for applying safe suction levels without the need for user adjustment or may constitute variable output regulating mechanisms. The fixed output regulating mechanisms are arranged for applying safe suction levels that are not prone to sticking or loss of regulation. Moreover, they solve problems associated with buzzing and vibration associated with prior art suction controls. The variable output regulating mechanisms of this invention are arranged for applying a predetermined level of suction and a different higher level, if necessary.

The devices of this invention can deliver a fixed output of suction pressure. There are a number of ranges that are preferred by clinicians. Oral and tracheal aspirations are performed in the range of 80-120 mm Hg, surgical; aspirations are performed at levels from 200-400 mm Hg and pediatric or neonatal suctioning is done at levels from 40-80 mm Hg.

Since the devices of this invention are designed to be disposable, it is contemplated that they will be labeled for single patient use to ensure that the functions and features are maintained. Moreover, devices constructed in accordance with this invention may make use of an indicating mechanism identifying that the unit is expired. Thus, as an additional precaution and aid to the clinical staff, it is anticipated to incorporate time based labeling that indicates expiration after a known period of use. This time based technology can be activated upon removal of the device from its package and will indicate prominently on the device that it has reached its useful life and should be discarded.

Without further elaboration the foregoing will so fully illustrate our invention that others may, by applying current or future knowledge, adopt the same for use under various conditions of service.

What is claimed is:

1. A device for controlling the level of suction to a patient in a hospital collection system having receptacle member coupled to a source of suction in the hospital collection system, the hospital collection system including a conduit arranged to be coupled to the patient to apply suction thereto, said device comprising a body, an inlet portion and an outlet portion, said inlet portion being arranged for connection to the conduit, said outlet portion being arranged for releasable connection to the receptacle member to couple the device to the source of suction of the hospital collection system to the patient, said body comprising a regulator for sensing and controlling the level of suction applied to said inlet portion and means for ensuring that when said device is subjected to excessive mechanical force said device operates in a predetermined manner.

2. The device of claim 1, wherein said predetermined manner comprises the outlet portion failing in said predetermined manner.

3. The device of claim 2 wherein said body comprises a necked down area and wherein when said outlet portion is subjected to the excessive force said outlet portion shears off at said necked down area of said body.

4. The device of claim 2 wherein said outlet portion is constructed so that the failure of said outlet portion in said predetermined fashion enables its ready removal from the wall plate.

5. The device of claim 1 wherein said predetermined manner comprises the device pulling out of the receptacle member.

6. The device of claim 1 wherein said body is formed of a plastic material.

7. The device of claim 1 wherein the receptacle member comprises a wall plate and wherein said device includes portions adapted to be inserted into said wall plate to releasably secure said device to said wall plate.

8. A device for controlling the level of suction to a patient in a hospital collection system having receptacle member coupled to a source of suction in the hospital collection system, the hospital collection system including a conduit arranged to be coupled to the patient to apply suction thereto, said device comprising a body, an inlet portion and an outlet portion, said inlet portion being arranged for connection to the conduit, said outlet portion being arranged for releasable connection to the receptacle member to couple the device to the source of suction of the hospital collection system to the patient, said body comprising a regulator for sensing and controlling the level of suction applied to said inlet portion and a fluidic dampening system to dampen oscillatory vibrations.

9. The device of claim 7 wherein said fluidic dampening system comprises a vent sized to dampen oscillatory vibrations.

10. A device for controlling the level of suction to a patient in a hospital collection system having receptacle member coupled to a source of suction in the hospital collection system, the hospital collection system including a conduit arranged to be coupled to the patient to apply suction thereto, said device comprising a body, an inlet portion and an outlet portion, said inlet portion being arranged for connection to the conduit, said outlet portion being arranged for releasable connection to the receptacle member to couple the device to the source of suction of the hospital collection system to the patient, said body comprising a regulator for sensing and controlling the level of suction applied to said inlet portion and means for ensuring that when disconnected from said receptacle member it cannot be reconnected thereto.

11. The device of claim 10 wherein the receptacle member comprises a wall plate and wherein said device includes portions arranged to be inserted into the wall plate, said device being arranged to be rotated to remove it from the wall plate, whereupon a portion of said device is displaced, said displaced portion preventing said device from being reconnected to the wall plate.
